(19) United States
(12) Patent Application Publication
(10) Pub. No.: US 2009/0221989 A1
(43) Pub. Date: Sep. 3, 2009

(54) SYSTEM AND METHOD FOR THE PREVENTION AND TREATMENT OF BACTERIAL AND FUNGAL INFECTIONS INCLUDING URINARY TRACT INFECTIONS (UTI) USING A HYPOHALOUS ACID COMPOSITION

(75) Inventors: Ramin Najafi, Novato, CA (US); Mansour Bassiri, Novato, CA (US); Lu Wang, Moraga, CA (US); Behzad Khosrovi, El Cerrito, CA (US)

Correspondence Address: FOLEY & LARDNER LLP, 975 PAGE MILL ROAD, PALO ALTO, CA, 94304, US

(73) Assignee: Novabay Pharmaceuticals Inc

(21) Appl. No.: 12/067,452

(22) PCT Filed: Sep. 21, 2006
(86) PCT No.: PCT/US06/36968

§ 371 Date: Oct. 29, 2008

(30) Foreign Application Priority Data
Sep. 21, 2005 (US) 60719439
Sep. 23, 2005 (US) 60720165

(51) Int. Cl. A61L 29/16; A61M 25/00; A61B 19/02; A61L 2/18; A61K 33/14; A61K 33/06; A61K 33/00; A61P 31/00

(52) U.S. Cl. 604/517; 604/265; 206/364; 422/37; 424/680; 424/679; 424/681; 424/682; 424/722

(57) ABSTRACT

Disclosed is a system that prevents the development of infection and biofilm establishment in medical devices in general, and in particular Urinary Tract Infections (UTI), including Catheter-Associated Urinary Tract Infections (CAUTI). The system comprises of a medical device (such as a catheter) and an antimicrobial composition containing an antimicrobial compound. A medical device delivers the composition both to the inside and/or outside portions of the device, as well as to the inside of the bladder itself and to the urethra. Reduction or elimination of the infection may be accomplished by irrigating the medical device, bathing the bladder, or irrigating the bladder with the composition.

This invention relates to a system that prevents the development of infection and biofilm establishment in medical devices in general, and in particular Urinary Tract Infections (UTI), including Catheter-Associated Urinary Tract Infections (CAUTI). The system comprises a medical device (such as a catheter) and an antimicrobial composition containing an antimicrobial compound. A medical device delivers the composition both to the inside and/or outside portions of the device, as well as to the inside of the bladder itself and to the urethra. Reduction or elimination of the infection may be accomplished by irrigating the medical device, bathing the bladder, or irrigating the bladder with the composition. Additionally, the medical device may be disinfected by such compositions prior to or during insertion through the urethral orifice. The medical devices described herein may also be stored in the compositions described herein. In addition to catheters or catheter-like devices other invasive medical devices such as pacemakers, heart valves, implantable devices, breast implants, intra-bone implants, stents, surgical plates, etc. may also be stored in the compositions described herein The materials detailed in this invention include compositions comprising hypohalous acids (HOCl or HOBr) or a hypohalous acid source. The relevant compositions have broad-spectrum, non-specific, rapid antimicrobial activity and are effective against planktonic microorganisms, and microorganisms associated with biofilm and encrustation.

### BACKGROUND OF THE INVENTION

Over 40% of hospital acquired infections are Urinary Tract Infections (UTIs) and most of these are Catheter-Associated Urinary Tract Infections (CAUTIs), occurring in patients with urinary catheters (Hashmi, Kelly et al. 2003). In fact, urinary catheters are the second most common cause of bacteremia (Maki and Tambyah 2001). Bacteremia is the presence of viable bacteria in the circulating blood, Various approaches designed to prevent CAUTI are in use; however, even in combination, they may only delay the onset of CAUTI but remain unable to prevent it.

Bacteriuria (the presence of bacteria in normally sterile urine) develops in 5% of catheterized patients per day (3-10%); almost all catheterized patients have bacteriuria by 30 days. Since asymptomatic bacteriuria may not be diagnosed initially, 10-25% of patients with bacteriuria develop UTI (Saint and Chenoweth 2003).

In 1-4% of patients with bacteriuria, the infection spreads into the kidney or bloodstream, leading to potentially lethal bacteremia (viable bacteria in the blood) (Saint and Chenoweth 2003).

The main reason for bacterial growth leading to CAUTI and bacteremia is the establishment of biofilm on the surfaces of the catheter (Morris, Stickler et al. 1999; Maki and Tambyah 2001; Tenke, Riedl et al. 2004; Trautner and Daouiche 2004). Biofilm is a matrix produced and inhabited by bacteria that leads to the development of microbial colonies encased in an adhesive, usually polysaccharide, material that is attached to a surface (e.g. the device). In addition to providing a reservoir of bacteria, biofilm can also result in catheter encrustation by crystal deposits created by the bacteria that, over time, can restrict flow through the catheter or even block it completely.

In one aspect, the system of this invention is effective by: (a) impeding bacterial build-up and (b) killing bacteria in and around the medical device, bladder and urinary tract. Such bacteria build up in and around the medical device and the bladder may include planktonic bacteria or bacteria in the form of biofilm, such as bacteria embedded in biofilm. Planktonic bacteria are free-floating bacteria, as opposed to sessile bacteria in biofilms. The system is also useful in preventing the formation of biofilm, killing bacteria embedded in biofilm, and removing biofilm. The system is also well tolerated, in particular, by inflamed or infected bladder tissue due to its low cytotoxicity. This unique combination of properties allows this system to effectively combat bacteriuria, thus limiting progression to CAUTI and bacteremia.

### FIELD OF THE INVENTION

The present invention relates to a system and methods for providing antimicrobial treatments. The system comprises a medical device (e.g. a catheter) and an antimicrobial composition. The methods comprise flushing, washing, instilling, irrigating and/or coating the medical device for the treatment, prevention or inhibition of infection by killing microbes and preventing microbial biofilm formation. The system may be provided in kits or trays for performing such treatment options.

The term “microbes” as used herein includes bacteria, fungi and viruses inhabiting areas around a medical device when used in patients.

The composition is useful in maintaining the medical device free from blockage and obstruction. The composition is also useful for treating, preventing and inhibiting infection including both inside and outside a patient's bladder. The medical device treated with a composition described herein is less likely to result in bacteriuria leading to urinary tract infection in patients receiving the medical device; one such device is a urinary catheter. Other medical devices include central venous catheters, intravascular catheters, such as cardiac catheters peritoneal dialysis catheters, dialysis shunts such as hemodialysis shunts, endotracheal tubes, surgical drain and device accessories such as ports.

Methods of using the pharmaceutical preparation of the invention in the management and maintenance of a medical device, such as a urethral catheter, are also disclosed in the present application.

### BACKGROUND OF THE INVENTION

A urinary catheter is a flexible tube system that is placed in the body to drain and collect urine from the bladder. Urinary catheters are used to drain the bladder during and after certain surgical procedures. Urinary catheters are also used to manage urinary incontinence and/or urinary retention in both men and women.

Depending on the underlying medical condition of the patient, a urinary catheter may be used (a) on an intermittent basis for just long enough to empty the bladder, (b) short term (hours or days, e.g. intra- and immediately post-operation), (c) longer term (few days to weeks, e.g. post-operation), or (d) continuous or chronic long term (30 days or more, e.g. spinal cord injuries (SCIs) and in Long Term Care Facilities (LTCFs)). An indwelling catheter that is left in place for a period of time is in general attached to a sterile container to collect the urine.

The most commonly used Foley indwelling catheter is a soft silicone or latex tube that is inserted into the bladder through the urethra to drain the urine, and is retained by a small balloon inflated with air or liquid. Urinary catheters come in a large variety of sizes, materials (latex, silicone, uncoated or coated with other materials such as silicone, hydrogel, antibacterial agents, etc.), and types (Foley catheter, straight catheter, Coude-tip catheter, etc.).

Catheters are generally placed into the bladder through the urethra, but in some cases, a suprapubic indwelling catheter is placed directly into the bladder through a surgically-prepared opening (stoma) in the abdomen above the pubic bone,

### Catheter-Related Complications:

Complications of indwelling catheter use may include catheter encrustation and obstruction, bacteriuria, urinary tract and/or kidney infections, which in turn may proceed to blood infections (bacteremia or septicemia). Intermittent catheter use may also result in bacteriuria (presence of bacteria in the urine) and subsequent urinary tract infection. Catheter encrustation stems from an infection caused by bacteria that produce urease; the increased activity of the urease results in an increased local pH and the formation of calcium and magnesium phosphate crystals. These crystals encrust the catheter and can cause partial or total blockage through of the catheter lumen (Stickler, Young et al. 2003).

### Definition of CAUTI:

Catheter associated urinary tract infection (CAUTI) is one of the most common nosocomial (hospital-acquired) infections in acute- and extended-care hospitals in the United Sates. It can affect the bladder and urethra, which are collectively known as the lower urinary tract.

The underlying cause of CAUTI is the formation of a pathogenic biofilm. Urease-producing bacteria colonize the catheter surface and create a biofilm community embedded in a polysaccharide matrix. The increased urease generates ammonia, which raises the pH of the biofilm and the urine; in this environment, hard crystals made of calcium and magnesium phosphate are formed and become embedded in the matrix (Stickler, Jones et al. 2003). There are few, if any, effective strategies to impede this process. Urethral catheters inevitably become colonized with attached microorganisms that are part of the biofilm community. Individuals develop bacteriuria at a rate of 3-10% per day; incidence reaches 100% in chronically catheterized individuals by 30 days (Trautner, Hull et al. 2005). The development of biofilm and crystalline encrustation of surfaces of urinary catheters has been demonstrated in a laboratory model using Proteus mirabilis (Stickler, Jones et al. 2003). Prophylactic bladder irrigation with antibiotics do not prevent colonization and lead to antibiotic resistance; prophylactic irrigation with hydrogen peroxide is also ineffective (Cravens and Zweig 2000).

Important routes of entry for bacteria into the bladder occur during the process of insertion of the catheter through the urethral orifice and by migration along the external surface of the catheter during movement of the catheter. Microorganisms found in urinary infections include Escherichia coli, enteric gram-negative rods such as Proteus, Enterobacrer and Klebsiella species, gram-positive bacteria, increasingly Candida yeast strains, and some enteric organisms such as Providencia and Pseudomonas (Hashmi, Kelly et al. 2003).

### DESCRIPTION OF RELATED ART

An effective treatment of CAUTI must essentially succeed in three areas: preventing/treating the infection, helping the catheter to resist encrustation and blockage due to the infection, and penetrating/eradicating the biofilm that allows the infection to thrive. A review of the literature, as summarized below, shows that there is presently no antimicrobial agent that solves all of these problems efficiently (Trautner and Darouiche 2004). The dominant problem in the strategies that have been attempted thus far is that flora resistant to the antimicrobial agents eventually reappear.

At present, the most effective strategy used to minimize CAUTI is the use of a closed drainage system; however, enhancements to this system are still needed to further minimize CAUTI. One such enhancement involves surface modification of the catheter material—that is, engineering the catheter material to make it inhospitable to CAUTI-causing bacteria. A review of catheters containing silver alloys in their matrix has shown they are only partially effective in reducing catheter-related bacteria (Saint and Chenoweth 2003). Urinary catheters impregnated with other antimicrobial agents have also been investigated to varying degree; devices with minocycline and rifampin (Darouiche, Smith et al. 1999), nitrofurazone (Maki and Tambyah 2001) and released gentamicin (Cho, Lee et al. 2001; Maki and Tambyah 2001) show some promise. However, with all of these agents, it is not clear whether prolonged use will result in the patient developing a resistance to the relevant bacteria (Saint and Chenoweth 2003). In fact, although some believe that surface modification shows more promise than instillation or irrigation (Tenke, Riedl et al. 2004), others believe that surface modification for preventing CAUTI has produced lackluster results at best (Trautner and Darouiche 2004).

That said, antimicrobial agents delivered systemically, instilled in the bladder, or used to irrigate the catheter have, thus far, shown to be ineffective for preventing CAUTI (Trautner and Darouiche 2004). A particular concern of catheter irrigation as a treatment for CAUTI is that for long-term catheterizations, the treatment will become ineffective because the bacteria and other flora that cause the CAUTI become resistant to said antimicrobial agent (Maki and Tambyah 2001; Saint and Chenoweth 2003; Trautner and Darouiche 2004). Studies using the antibiotic neomycin and independently the antimicrobial povidone-iodine for irrigation have shown no benefit for treating CAUTI (Hashmi, Kelly et al. 2003).

The use of bladder irrigation or instillation has been recommended to prevent debris and stone formation as well as infection (Galloway 1997). Urinary catheters, and Foley catheters in particular, are highly susceptible to encrustation and blockage from crystals generated by the local bacteria (Stickler, Young et al. 2003); the use of an antimicrobial solution to irrigate the catheter may have some success in preventing encrustation and blockage. Laboratory experiments using triclosan as the antimicrobial agent have show promise in preventing encrustation (Stickler, Jones et al. 2003); however, long term use of this agent in the body may result in the emergence of resistant bacteria. Similarly, although there has been some success using chlorhexidine solutions for this purpose (Baillie 1987; Pearman, Bailey et al. 1991), it is not practical for long term use because the bacteria develop resistance to the chlorhexidine (Baillie 1987). Additionally, breaking the closed drainage system of the catheter increases risk of infection and physical injury to the patient (Galloway 1997; Cravens and Zweig 2000).

Yet another consideration in using antimicrobial agents in urinary catheters is whether or not the agent will be able to penetrate and dislodge biofilm. The use of saline for irrigating catheters has little to no effect in reducing bacteriuria and dislodging biofilm (Muncie, Hoopes et al. 1989). Thus far, the use of antimicrobial agents (as ointments and lubricants, in collection bags, impregnated within the catheter material, and with bladder instillation or irrigation) has also resulted in a failure to treat biofilms (Donlan and Costerton 2002; Tenke, Riedl et al. 2004).

### DESCRIPTION OF THE INVENTION

The system provided herein comprises a medical device (such as a catheter) and an antimicrobial compound. Together, they provide antimicrobial treatment options that do not have the undesirable properties of (a) inducing bacterial resistance and (b) significant toxicity. The antimicrobial compound can either be incorporated or embedded into the device material such that a hypohalous acid is generated or activated on contact with aqueous fluids. In another aspect, the antimicrobial compound can be added to an aqueous solution and be used as part of the resulting antimicrobial composition.

The system provided includes an embodiment wherein the medical device is a Central Venous Catheter (CVC). This type of catheter is placed into a large vein in the neck, chest, or groin. While all catheters can introduce bacteria into the bloodstream, CVCs can also cause Staphylococcus aureus sepsis and Staphylococcus epidermidis sepsis.

The system provided also includes an embodiment wherein the medical device is a Peritoneal Dialysis Catheter. In case of kidney failure, peritoneal dialysis is used for removing waste such as urea and potassium from the blood, as well as removing excess fluid, Peritoneal dialysis requires access to the peritoneum, a natural semipermeable membrane surrounding the intestine. This access breaks normal skin barriers, and as people with renal failure generally have a slightly suppressed immune system, infection is a relatively common problem.

Peritoneal dialysis is typically done in the patient's home and workplace, but can be done almost anywhere; a clean area to work, a way to elevate the bag of dialysis fluid and a method of warming the fluid are all that is needed. The main consideration is the potential for infection with a catheter; peritonitis is a commonest serious complication, and infections of the catheter exit site or “tunnel” (path from the peritoneum to the exit site) are less serious but more frequent. Because of this, patients are advised to take a number of precautions against infection.

Peritoneal dialysis is a method for removing waste such as urea and potassium from the blood, as well as excess fluid, when the kidneys are incapable of this (i.e. in renal failure). It is a form of renal dialysis, and is thus a renal replacement therapy. Peritoneal dialysis works on the principle that the peritoneal membrane that surrounds the intestine, can act as a natural semipermeable membrane (see dialysis), and that if a specially formulated dialysis fluid is instilled around the membrane then dialysis can occur, by diffusion. Excess fluid can also be removed by osmosis, by altering the concentration of glucose in the fluid. Dialysis fluid is instilled via a peritoneal dialysis catheter, (the most common type is called a Tenckhoff Catheter) which is placed in the patient's abdomen, running from the peritoneum out to the surface, near the navel. Peritoneal dialysis catheters may also be tunnelled under the skin and exit alternate locations such as near the rib margin or sternum (called a presternal catheter), or even up near the clavicle. This is done as a short surgery. The exit site is chosen based on surgeon's or patient's preference and can be influenced by anatomy or hygeine issues. More details can be found in http://en.wikiedia.org/wiki/Peritoneal_dialsis or in Merck's Manual of Medical Information (hereinafter “MMOMI”), Home Edition, 1997, Editor-in-Chief Robert Berkow, M.D. pp. 600, 656-658.

The system provided includes an embodiment wherein the medical device is a Hemodialysis Shunt. The 3 most common types are an intravenous catheter, an arteriovenous (AV) Cimino fistula, or a synthetic graft. In all three cases, two tubes (or one tube with two lumen) are required to first remove blood to be cleansed and then to return clean blood to the body. Since hemodialysis requires continuous access to the circulatory system through the skin, patients undergoing hemodialysis have a portal of entry for microbes, which could lead to septicemia or an infection affecting the heart valves (endocarditis) or bone (osteomyelitis). More details can be found in Reference. http://en.wikipedia.org/wiki/Hemodialysis and MMOMI, pp. 654-657.

The system provided includes an embodiment wherein the medical device is an endotracheal tube (ETT). ETTs are put in the mouth and then down into the trachea (the airway) for the purpose of airway management and lung ventilation. These ETT's are at high risk for causing ventilator-associated pneumonia (VAP) in patients. VAP is a subset of hospital-acquired pneumonia and occurs after at least 48 hours of intubation and mechanical ventilation. There are several bacteria which are particularly important causes of VAP because of their resistance to commonly used antibiotics. More details can be found in Reference: http://en.wikipedia.org/wiki/Ventilator-associated_pneumonia.

The system provided includes an embodiment wherein the medical device is a surgical drain. A surgical drain is a tube used to remove pus, blood or other fluids from a wound or larger pleural effusions. Drains inserted after surgery help the wound to heal faster. Details can be found in MMOMI, pp. 225-227, 935-936 and 171.

The system provided includes an embodiment wherein the medical device is an accessory to a medical device susceptible to bacterial infection, such as a port.

The use of the antimicrobial compounds described herein may be useful as being an effective treatment or prevention of various bacterial or fungal infections, including Urinary Tract Infection (UTI) and in particular of Catheter-Associated Urinary Tract Infection (CAUTI) in these critical areas: minimizing the opportunity for bacterial biofilm formations that would allow the infection to thrive and potentially cause bacteriuria in catheterized patients, penetrating/eradicating or reducing the biofilm that is able to form, and helping the catheter to resist encrustation and blockage due to the infection and subsequent biofilm formation. The system may also be useful in treating and preventing other microbial infections, such as viral, yeast or fungal infections, in particular those associated with bacterial infections. One of the treatment options is to administer one of the compositions described herein through a catheter to a patient where previously saline or vinegar was used and bacterial infection had occurred.

The antimicrobial compound employed in the practice of the present invention is one that is not classified as an antibiotic. For purposes of the present invention, the term “antibiotic” is defined as a chemical substance produced by microorganisms, or synthetic or semi-synthetic analog, or a derivative of such a chemical substance, that can inhibit or destroy susceptible microorganisms (e.g. penicillin).

It is an object of the present invention to avoid the overuse of these traditional antibiotics, although they may, if desired, be used systemically in conjunction with the system of the invention. Compositions of antimicrobial compounds and antimicrobial compositions are provided for use in flushing and coating medical devices, especially catheters and ports.

The preferred medical devices of this invention are urinary catheters as described herein.

Urinary catheters consist of a tube that is inserted through the urethra into the bladder. In men, it is inserted through the tip of the penis, and in women, it is inserted through the meatus.

The best known catheter is the double-lumen Foley catheter, a device often employed with hospital patients recovering from surgery. The tip of the Foley catheter is inserted until it enters the bladder. An inflatable, small, bi-lateral balloon near the tip holds the catheter in place when inflated. The tip of the tube has openings to allow flow of urine into a container for collection. A side port, for example a “T” junction, may be introduced into the catheter pathway in order to facilitate repeated instillation and irrigation while minimizing avenues for added infection. These catheters can be flushed using intermittent back flow (that is, irrigation of the treatment composition from the port opening back up the catheter into the bladder).

In cases where the need for flushing or rinsing of the bladder is anticipated, for example to remove blood and debris after surgery, a triple-lumen Foley catheter may be used instead. This catheter-type has an additional lumen through which fluid from a reservoir can be provided into the bladder and flushed out through the main lumen together with urine into a container. These catheters can be flushed using continuous flow. Generally, the reservoir will be configured to secure the catheter in place when the device is inserted into the bladder of a patient,

Typical catheters used in accordance with the treatment described herein are disclosed in U.S. Pat. No. 4,245,639 and U.S. Pat. No. 4,337,775. These catheters have drainage means (for example, a cannula) and means for holding the drainage means in place in the bladder of a patient (for example, an inflatable balloon). The drainage and holding means have inner and outer surfaces that may be exposed to bacterial biofilm formation.

Catheters are generally placed into the bladder through the urethra, but in some cases, a suprapubic indwelling catheter is placed directly into the bladder through a surgically prepared opening (stoma) in the abdomen above the pubic bone.

### The Antimicrobial Composition:

In one aspect of the invention, there is provided a method for treating a medical device and/or surrounding tissue with a biocidally (i.e. ability of inactivating pathogens) effective amount of an antimicrobial composition. In another aspect, there is provided a method of treating, inhibiting, reducing or preventing infection associated with the use of the medical device before or after it has been inserted in a patient.

In another aspect, there is provided an antimicrobial treatment system comprising

(a) a medical device, optionally including an accessory to the device, for implantation or insertion into a patient at risk of, or affected by, a microbial infection; and

(b) an aqueous antimicrobial composition comprising
(1) a composition comprising an antimicrobially effective amount of hypochlorous acid HOCl, a source of hypochlorous acid, hypobromous acid HOBr, or a source of hypobromous acid; and
(2) at least one halide salt selected from the group consisting of sodium chloride, sodium bromide, potassium chloride, potassium bromide, magnesium chloride, magnesium bromide and mixtures thereof;
(3) the halide salt concentration ranging from at least about 4 to about 12 g/l of the aqueous composition;
(4) a pH from about 3 to about 6; and optionally
(5) a constituent member selected from the group consisting of buffering agents, calcium and magnesium chelating agents, biologically acceptable acids and/or salts thereof that are compatible with the antimicrobial treatment system, and mixtures thereof to maintain the pH at the range between about 3 and 6
   in order to prevent or treat colonization of the device by microbes, buildup of microbial biofilm on the device, or blockage of the device by the microbial biofilm. The term “halide salt” and the term “saline component” are used interchangeably herein to reflect the fact that the compositions described herein aim to achieve biologically or physiologically acceptable salt concentrations. The term “blockage of the device” includes encrustation.

In one embodiment, the concentration of biologically acceptable acids and/or salts thereof is about 1 mM to about 100 mM.

In a particular variation of the above system, the antimicrobially effective amount of the hypohalous acid (hypochlorous or hypobromous acid) derived from the hypohalous acid or the hypohalous acid source is present at a concentration of about 0.1 mM to about 75 mM in the aqueous composition. In one variation, the medical device is a catheter for insertion into the bladder of a patient at risk of, or affected by, a bacterial, fungal or viral infection in or around the bladder and/or infections in the patient's bloodstream. In another variation of the above, the patient is at risk of, or affected by, bacteriuria or bacteremia Urinary Tract Infections (UTI), and/or Catheter-Associated Urinary Tract Infections (CAUTI). In a particular variation of the system, the hypohalous acid or hypohalous acid source concentration is about 2 mM to about 20 mM in the composition. In one variation, the hypohalous acid is hypochlorous acid.

In one aspect of the above system, the pH is about 3.3 to about 5.5. In one variation, the pH is about 3.5 to about 5. In a particular variation, the halide salt concentration is about 7 to about 10 g/l. In another variation, the halide salt concentration is about 9 g/l. In another aspect of the above system, the buffering agent composition is selected to maintain the pH between about 3.3 to about 5.5. In one variation, the buffering agent composition is selected to maintain the pH between about 3.5 to about 5.0. In another variation of the above system, the constituent member concentration ranges from about 1 to 100 mM. In a particular variation, the chelating agent concentration is selected to chelate up to about 10 mM of a member selected from the group consisting of calcium, magnesium or mixtures thereof. In yet another variation, the chelating agent concentration is selected to chelate up to about 5 mM of a member selected from the group consisting of calcium, magnesium or mixtures thereof. In another variation of the above, the chelating agent concentration is selected to chelate up to about 2 mM of a member selected from the group consisting of calcium, magnesium or mixtures thereof. In a particular variation, the chelating agent concentration is selected to chelate up to about 1 mM of a member selected from the group consisting of calcium, magnesium or mixtures thereof. In another embodiment, the system of the invention comprises about 2 mM to about 20 mM of the hypohalous acid or the hypohalous acid source, the pH is about 3.3 to about 5.5; the halide salt concentration is about 7 to about 10 g/l of the composition; and the buffering agent concentration is about 0 or about 1 mg/l to about 100 mg/ml; the chelating agent concentration is about 0 mg/ml or about 1 mg/ml to about 100 mg/l; and the biologically acceptable acid and/or salt concentration is about 0, or about 1 to 100 mg/ml.

In another aspect, there is provided a catheter treated with an aqueous antimicrobial composition for the treatment or prevention of bacteriuria or CAUTI or associated fungal or viral infections, the aqueous antimicrobial composition comprising: (A) an antimicrobially effective amount of at least one hypohalous acid (HOHal, wherein Hal is chloro or bromo), or a hypohalous acid source; (B) at least one halide salt selected from the group consisting of sodium chloride, sodium bromide, potassium chloride, potassium bromide, magnesium chloride, magnesium bromide and mixtures thereof, the saline component (halide salt) concentration ranging from at least about 4 to about 12 g/l of the composition; (C) wherein the pH of the composition is about 3 to about 6; and (D) the antimicrobially effective amount of the hypohalous acid derived from the hypohalous acid or the hypohalous acid source is about 0.1 mM to about 75 mM of the composition; and optionally (E) a constituent member selected from the group consisting of buffering agents, calcium and magnesium chelating agents, biologically acceptable acids and salts thereof that are compatible with the antimicrobial treatment system, and mixtures thereof to maintain the pH between about 3 and 6.

In another aspect, there is provided a catheter treated with an aqueous antimicrobial composition for the treatment or prevention of bacteriuria or CAUTI or bacteremia or associated fungal or viral infections, the composition comprising; (A) an antimicrobially effective amount of at least one hypohalous acid (HOHal), wherein Hal is chloro or bromo, or a hypohalous source; (B) at least one saline component (halide salt) selected from the group consisting of sodium chloride, sodium bromide, potassium chloride, potassium bromide, magnesium chloride, magnesium bromide and mixtures thereof, the halide salt concentration ranging from at least about 4 to about 12 g/l of the aqueous composition; (C) wherein the pH of the composition is about 3 to about 6; and (D) the antimicrobially effective amount of the hypohalous acid or the hypohalous acid derived from the hypohalous acid source ranging from about 0.1 mM to about 75 mM in the aqueous composition; and (E) a constituent member selected from the group consisting of buffering agents, calcium and magnesium chelating agents, biologically acceptable acids and salts thereof that are compatible with the antimicrobial treatment system, and mixtures thereof to maintain the pH at the range between about 3 and 6 and in order to prevent or treat colonization of the device by microbes, buildup of microbial biofilm on the device, or blockage of the device by the microbial biofilm.

In another aspect, there is provided a method of treating, inhibiting or preventing an antimicrobial infection in or near a medical device before or after said device has been inserted into a patient or a method of treating, inhibiting or preventing bacterial, viral or fungal infection in a patient after said device has been inserted into a patient which comprises the following treatment steps in isolation or in combination: (a) contacting the device with a composition comprising elements (A) through (E) of the above aspects, prior to insertion into a patient or after removal from a patient; (b) washing, bathing or flushing the device with a composition comprising elements (A) through (E) of the above aspects, prior to insertion into a patient or after removal from a patient; (c) irrigating the device with a composition comprising elements (A) through (E) of the above aspects, after insertion into a patient, in order to prevent or treat colonization of the device by microbes, buildup of microbial biofilm on the device, or blockage of the device by the microbial biofilm including encrustation on the device; or (d) instilling through the device a composition comprising components elements (A) through (E) of the above aspects, into the bladder of a patient to treat or prevent a fungal or bacterial infection of the lining of the bladder.

In another aspect, there is provided a method comprising treating or contacting the medical device with a biocidally-effective amount of an aqueous antimicrobial composition, the composition comprising:

(A) an antimicrobial compound, comprising
(1) at least one hypohalous acid (HOHal), Hal is Cl or Br or a hypohalous acid source;
(2) the hypohalous acid concentration ranging from about 0.1 mM to about 75 mM in the composition; and

(B) an aqueous solution, comprising
(1) at least one saline component (halide salt) selected from the group consisting of sodium chloride, sodium bromide, potassium chloride, potassium bromide, magnesium chloride and magnesium bromide;
(2) the pH of the composition ranging from about 3.0 to about 6.0;
(3) the saline component (halide salt) concentration ranging from at least about 4 to about 12 g/l of the aqueous composition; and optionally
(4) other constituents including acids, buffering and chelating agents, either organic or inorganic.

In another aspect, there is provided an aqueous antimicrobial composition for the treatment or prevention of a microbial infection in patient, said composition comprising (a) an antimicrobially effective amount of a hypohalous acid (HOHal, wherein Hal is chloro or bromo) or a hypohalous acid source; (b) at least one saline component (halide salt) selected from the group consisting of sodium chloride, sodium bromide, potassium chloride, potassium bromide, magnesium chloride, magnesium bromide and mixtures thereof; the saline component (halide salt) concentration is at least about 4 to about 12 g/l of the aqueous composition; (c) a pH of about 3 to about 6; and (d) a constituent member selected from the group consisting of buffering agents, calcium and magnesium chelating agents, biologically acceptable acids and/or salts thereof that are compatible with the antimicrobial composition, and mixtures thereof to maintain the pH at the range between about 3 and 6.

In another aspect, the method comprises administering the above aqueous solution to the patient using the medical device. In one particular variation, the medical device is a catheter.

A hypohalous acid source is a composition that, under the appropriate condition, has the ability to release a hypohalous acid. The hypohalous acid source may be a physical composition, for example, a carrier of hypohalous acid that is compatible with the hypohalous acid and not oxidizable by the hypohalous acid. Such a carrier may be a non-oxidizable material, such as a cloth that may be used in conjunction with the system described herein, for example for the purpose of cleaning urethral openings. Another hypohalous source may include non-oxidizable microcapsules that will release a hypohalous acid when in contact with water or aqueous systems or solutions, such as a bodily fluid. Another chemical hypohalous acid source may be a hypohalous salt, such as sodium or calcium hypochlorite which releases hypohalous acid when placed in contact with an acid. Another hypohalous source may be hypohalous acid precursor or prodrug which releases a hypohalous acid when contacted with water or aqueous systems or solutions, such as a bodily fluid. An example of such a hypohalous precursor or prodrug is trichloroisocyanuric acid (Symclosene) or one of its derivatives, for example a sodium salt thereof. The preferred hypohalous source is a hypohalous acid and most preferably hypochlorous acid.

In another aspect, this disclosure describes an antimicrobial composition for use with medical devices as discussed herein.

A preferred device treatment or medical treatment of a patient uses an antimicrobial composition containing the antimicrobial compound hypochlorous acid.

The devices or treatments are preferred where the hypohalous acid concentration ranges from about 2 mM to about 20 mM in the composition. In one variation, the hypohalous acid concentration ranges from about 5 mM to about 15 mM, or about 8 mM to about 12 mM. In another variation, the hypohalous acid concentration ranges from about 2 mM to about 8 mM, or about 2 mM to about 5 mM. In another variation, the hypohalous acid concentration ranges from about 10 mM to about 20 mM, or about 15 mM to about 20 mM. Amounts are provided in mM, which equals millimoles per liter.

Concerning the saline component (halide salt), the preferred inorganic salt is sodium chloride at a concentration of about 0.4 to about 1.2% by weight NaCl which is about four-tenth to slightly higher than normal or isotonic saline solution According to Parker's McGraw-Hill Dictionary of Scientific and Technical Terms, S. P. Parker, editor, Fifth Edition, “normal saline”, “physiological saline”, “physiological salt solution” are defined as a “solution of sodium chloride in purified water, containing 0.9 grams of sodium chloride in 100 milliliters; isotonic with body fluids.” For different halide salts such as lithium halides, potassium halides, and the like, the concentration of the salt in making up an isotonic solution may differ from the concentration of sodium chloride in an aqueous solution in order to maintain the desired osmolarity of the solution of the invention. In yet another aspect of the invention, the inorganic salt in the aqueous solution is at a concentration of about 0.7 to about 1.0 by weight %. In a variation of the above, the inorganic salt is sodium chloride.

More effective devices may be treated with a composition where the saline component concentration ranges from about 7 to about 10 g/l of the composition. Likewise, in the most effective antimicrobial treatment options patients are treated with antimicrobial compositions where the halide salt concentration will be from about 7 to about 10 g/l, with 9 g/l being most preferred.

The preferred pH for the treatment ranges from about 3.3 to about 5.5, and even more preferred, from about 3.5 to about 5.0. Depending on its use the pH may be from about 3.5 to about 4.0, 3.8 to about 4.3; 4.0 to about 4.5; 4.3 to about 4.8 or at about 4.5 to about 5.0, or at about 4.8 to about 5.3, or about 5.0 to about 5.5. The pH may be at any pH range within the broad pH range from about 3.0 to about 6.0. For example, for patients with the risk of encrustation forming around the tip of the catheter more acidic pH ranges would be preferred to counteract crystal deposits from calcium or magnesium phosphate crystals.

As explained earlier, bacteria in areas in and around a medical device or the bladder produce urease, an enzyme which hydrolyzes urea to carbon dioxide and two equivalents of ammonia. The hydrolysis raises the pH of the urine. As a result of the increased pH, the formation of calcium and magnesium phosphate deposits is favored, which may result in encrustation of the tip of a catheter.

Buffer Systems: To counteract the increase of the pH, appropriate buffer systems may be used to maintain the pH at a lower range. The selection of the optimum buffer systems and buffer conditions and buffer concentrations is known to a person skilled in the art. It may among other factors, depend on the pH of the urine, the amount of urea in the urine, the degree and kind of bacterial infection, etc. however, in general, buffer amounts may be present in the antimicrobial compositions herein described in an amount to maintain the pH in and around the catheter and the bladder of the patient between 3 and 6.

Examples of buffer systems comprising electrolyte solutions include well known buffer systems such as Clark and Lubs solutions, pH 2.2-4.0 (Bower and Bates, J. Res Natn. Bur. Stand. 55, 197 (1955)); β,β-dimethylglutaric acid-NaOH buffer solutions, pH 3.2-7.0 (Stafford, Watson, and Rand, BBA 18, 318 (1955)); sodium acetate-acetic acid buffer solutions, pH 3.7-5.6; succinic acid-NaOH buffer solutions, pH 3.8-6.0 (Gomeri, Meth. Enzymol. 1, 141 (1955)); sodium cacodylate-HCl buffer solutions, pH 5.0-7.0 (Pumel, Bull. Soc. Chim. Biol. 30, 129 (1948)); Na₂HPO₄—NaH₁₂PO₄ buffer solutions, pH 5.8-7.0 (Gomeri and Sorensons, Meth. Enzmol. 1, 143 (1955)); potassium biphthalate/HCl, pH 3.0 to 3.8; potassium biphthalate/NaOH pH 4.0-6; KH₂PO₄ NaOH, pH 6.07.0 (see OECD Guideline for Testing Chemicals “Hydrolysis as a Function of pH,” Adopted 12 May 1981, 111, pp. 10-11).

Acids, Esters and Salts: A preferred acid is one that is at a biologically safe concentration and is biologically compatible with the antimicrobial compound. The acid is a member of the group selected from acetic acid, benzoic acid, propionic acid, oxalic acid, hydrochloric acid, phosphoric acid, sulfuric acid, boric acid, diethylenetriamine pentaacetic acid, and esters of p-hydroxybenzoic acid (Parabens), or the biologically acceptable salt form of the acid may be a member of the group selected from potassium citrate, potassium metaphosphate, sodium acetate, and sodium phosphate.

Chelating Agents: The antimicrobial composition may also comprise a biologically acceptable, and in the presence of the antimicrobial compound, stable chelating agent that prevents encrustation of the device (e.g. by insoluble salts of Ca²+ or Mg²+). Other examples include malic acid and maltol, or their derivatives or mixtures thereof.

Depending on the nature of the constituents, each of these constituents may serve multiple functions. For example, a single constituent may have acidic, buffering and/or chelating properties. The preferred concentration ranges for other constituents is 1 to 100 mM.

Because the catheter surface plays an important role in biofilm formation, preferred device surfaces have increased hydrophilicity which provide a softer surface for tissue contact and reduced susceptibility of CAUTI and bacteriuria. Increased surface hydrophilicity may be effected by hydrogel-coating, for example, with polyvinyl pyrrolidone and polyethylene glycol.

Alternatively, the antimicrobial compound (i.e., the hypohalous source) can either be incorporated or embedded into the device material such that the hypohalous acid is generated or activated on contact with aqueous fluids. Furthermore, the compound may be allowed to slowly diffuse into the surrounding space. Alternatively, it could be present in an inactive state and be activated by a chemical reaction with a substrate that it supplied to the catheter in an aqueous solution.

Optionally, a patient may be treated systemically with broad spectrum or specific antibiotics at the same time, in combination with the methods of the present invention.

In some instances the device comprises the antimicrobial composition contained in a reservoir connected with the device (see FIG. 3). Commonly the reservoir is elevated above the position of the device itself, for example a hanging bottle.

The device may be configured in a way wherein the reservoir is in an antimicrobial composition dispensing device in a drainage receptacle receiving a biological fluid. The drainage receptacle may be configured in such a way that multiple dispensing devices could be placed into the drainage receptacle, perhaps when emptying the urine from the receptacle. Preferred devices will have the dispensing device in the lower portion of the drainage receptacle and the antimicrobial composition will be dispensed from the dispensing device into the receptacle.

The uses of catheters that benefit most from the treatment described herein are the uses of indwelling catheters, for example, a Foley catheter. Alternatively, the catheter may also be an intermittent catheter.

Likewise, patients that benefit from the treatment described herein are patients that are suffering from infections that may be both related and unrelated to the use of catheters. Examples include interstitial cystitis caused or aggravated by bacterial infections, or fungal cystitis, underactive bladder diseases, particularly caused by neurological injuries or disorders, overactive bladder diseases, lack of bladder control, such as urinary incontinence patients, patients suffering from CAUTI, bacteriuria, or urethral injuries, etc.

The devices herein described may be treated with an above-described antimicrobial composition prior to insertion through the urethral orifice. Some device treatment options include irrigation, flushing, rinsing or washing of the device. Some treatment options include irrigation and instillation using the compositions described herein into a patient's bladder.

### Procedures for the Method of Treatment:

The method of treating, inhibiting, reducing or preventing infection in or near a medical device before or after the device has been inserted in a patient, and the method of preventing or treating infection in a patient after the device has been inserted in a patient comprises the following individual treatment steps in isolation or in combination:

(a) contacting the device with the above defined antimicrobial composition prior to insertion in a patient or after removal from a patient;

(b) washing, bathing or flushing the device with the above defined antimicrobial composition prior to insertion in a patient or after removal from a patient;

(c) irrigating the device with above defined antimicrobial composition after insertion in a patient, in order to prevent or treat colonization of the device by microbes, buildup of microbial biofilm on the device, or blockage of the device by the microbial biofilm on the device; or

(d) instilling through the device an antimicrobial composition into the bladder of a patient to treat or prevent a fungal, viral or bacterial infection of the lining of the bladder or urethra.

The above individual treatment steps are described below.

The treatment of a patient to treat, inhibit or prevent microbial infection should use a sufficient amount of a solution comprising a composition as described herein. A sufficient amount means a dose range between 1 and 100 ml for instillation and 10 to 1,000 ml for irrigation with a hypohalous concentration as described herein for one treatment procedure (for example, irrigation or instillation), or as deemed necessary for the particular application. It is self-evident that in case of severe infection the procedure may have to be repeated to maximize the antimicrobial action.

The present invention also relates to a device treated with the above described antimicrobial composition or a method of treating, inhibiting, reducing or preventing infection in or near a medical device before or after said device has been inserted in a patient comprising (a) treating or contacting the device, or the patient through the device, with a biocidally effective amount of the above described antimicrobial composition, or (b) administering to the device or to the patient through the device the above described antimicrobial composition. In another aspect, the present invention also relates to a method of treating, inhibiting, reducing or preventing infection in or near a medical device before or after said device has been inserted in a patient or a method of treating, inhibiting or preventing infection in a patient comprising (a) treating or contacting the device, or the patient through the device, with a biocidally effective amount of the above described antimicrobial composition, or (b) administering to the device or to the patient through the device the above described antimicrobial composition

The amount of solution of the antimicrobial composition used for the treatment of a catheter device should be enough to fill it. Such devices, typically have internal volumes in the range of about 1 to 3 mL. However, the volume will, of course, vary with the length and diameter of the tubing of the device, which may depend on the individual patient. Larger volumes (e.g. 20-100 ml) of the antimicrobial composition as described herein may be needed for procedures such as bladder instillation.

### Pre-Treatment Using the Antimicrobial Composition:

Although the medical treatment options described herein and the treated devices of the present invention are primarily concerned with introducing the antimicrobial compositions into catheters that are already in place, those skilled in the art will appreciate that contacting the patient's body at and around the site of insertion can aid in the elimination of sites for bacterial growth. Thus, patients can be treated and the surfaces of medical devices, such as catheters, can be pre-treated by the compositions of the present invention to prevent bacteriuria and thereby prevent the infection that may ensue. In one method, the medical device can be treated with a composition initially and then, after insertion, with repeated periodic antimicrobial treatment options described above. The pre-treatment of the device can also be effected by irrigation. It may also advantageous to pre-treat the orifice of the patient before using the catheter.

### Packaging:

The invention also relates to kits or trays that include the above described antimicrobial compositions that are useful for the treatment methods described herein. For example, such kits or trays may comprise a closed sterile catheter syringe pre-filled with the antimicrobial composition for catheter insertion, irrigation or instillation purposes. The trays or kits may include lubricant, prepackaged disinfectant supplies, additional prepackaged antimicrobial composition, pre-packaged alcohol wipes etc. In addition, the kits or trays may contain instructions how to use the kits or trays in the treatments described herein. The invasive devices may also be stored in the antibacterial compositions described herein prior to implantation or insertion into the patient.

### Microorganisms Treated:

Use of catheters treated with the antimicrobial compositions described herein reduces bacteriuria caused by, but not limited to, the following microorganisms (bacteria, viral and fungi): Staphylococcus aureus, Staphlylococcus saprophyticus, Staphylococcus epidermidis, and other Staphylococcus species, Escherichia coli, Pseudomonas aeruginosa, Proteus mirabilis, Providenicia stuartii, Pseudomonas sp. Enterococci, Proteus species, Klebsiella pneumoniae, Enterobacter species, Candida species, Candida galabrata, Candida albicans, Serratia marcescens, Citrotacter spp., Morganella morganii, Enterococcus faecalis, Stenotrophomonas species, Clostridium difficile, Lactobacllus species, and other uropathogenic microorganisms, adenovirus and herpes.

Treatment of a catheter, or antimicrobial treatment of a patient in accordance with this disclosure, includes treatment of the devices described, such as a catheter with the above described antimicrobial composition or administering such compositions to a patient through a catheter device. Such treatments include treatment of the catheter prior to its use and also treatment of the catheter while inserted in a male or female patient, adult, or child. The treatment includes any form of contact of a composition described herein with the catheter and antimicrobial treatment with the compositions described herein through administration to a patient. Non-limiting examples of such treatments include rinsing, washing, flushing, instillation and irrigation. The treatment also includes inflation of the balloon of a catheter with the antimicrobial composition. The treatment may also include bathing a patient's bladder with the compositions described herein.

### Example 1

Some representative compositions for use with a catheter include:

### Composition A:

### 2 mM HOCl

0.9% salt (150 mM)
pH 4

### Composition B3:

### 2 mM HOCl

0.4% salt (150 mM)
pH 3.5

### Composition C:

### 6 mM HOCl

0.9% salt (150 mM)
pH4
10 mM sodium acetate-acetic acid

### Composition D:

### 6 mM HOCl

0.9% salt (150 mM)
pH 5
15 mM malic acid

### Composition E.

### 10 mM HOCl

0.9% salt (150 mM)
pH6
20 mM phosphate

### Example 2

### Inserting a Catheter Through the Urethra in Women and Men

The following is a description of a general procedure for inserting a catheter and for using the antimicrobial composition. Assuming that a person skilled in the art is proficient in sterile techniques and in working with catheters, including dealing with obstructions and knowing when to call a physician, nurse or medical specialist for assistance, only those steps relevant to this invention are described. The other steps of the procedure (for example, hand cleansing or sanitization, lubrication of the catheter, inflating the balloon of the catheter once the catheter is in place), safeguards (for example, the use of sterile gloves and how to use them), instructions to the treated patient (for example, breathing or relaxation instructions) are familiar to physicians or nurses.

Use 5-100 ml of the antimicrobial Composition C (as described in Example 1) to clean the urethral opening.

Throughout the process of insertion, gently push antimicrobial composition up through the catheter, so that the urethra gets disinfected prior to coming into contact with the catheter.

Use 1-20 ml of the antimicrobial Composition C to wet the catheter as it is being inserted through the urethra into the bladder.

### Example 3

### Opening a Partially Obstructed (Encrusted) Urinary Catheter

The following is an example of a catheter irrigation procedure to improve flow through a partially obstructed catheter. The catheter is irrigated with the composition to remove an encrustation at the tip of the catheter (plug) so that the urine can drain from the bladder.

Irrigation of a catheter in accordance with the invention may constitute a procedure to open a plugged urinary catheter with the above described antimicrobial composition. Assuming that a person skilled in the art is proficient in sterile technique and in working with catheters, including dealing with obstructions and knowing when to call a physician, nurse or medical specialist for assistance, only those steps relevant to this invention are described. The other steps of the procedure (how to deflate the balloon), safeguards (for example, the use of sterile gloves and how to use them) instructions to the treated patient (for example, breathing or relaxation instructions) are familiar to physicians or nurses.

The following instructions can be used for an irrigation procedure with the composition disclosed herein:

Draw up 1 to 100 mL of the antimicrobial Composition A (as described in Example 1) into a syringe.

After disconnecting the catheter from the drainage tubing, insert the syringe with the antimicrobial composition into the catheter.

Gently push on the plunger of the syringe to slowly push the composition into the catheter. Do not force the composition into the catheter.

If the composition does not flow easily into the catheter, gently pull back on the plunger to aspirate (withdraw) fluid, using very little force.

After inserting the antimicrobial composition into the catheter, remove the syringe from the catheter and insert the connecting tubing.

Check the tubing after reconnecting to see if urine is flowing. If no urine is flowing after 10 to 15 minutes, repeat the irrigation process.

### Example 4

### Bladder Instillation Procedure

The following instructions can be used for an instillation procedure with the composition disclosed herein for a patient. Assuming that a person skilled in the art is proficient in sterile technique and in working with catheters, including dealing with obstructions, only those steps relevant to this invention are described. The other steps of the procedure, safeguards (for example, the use of sterile gloves and how to use them) instructions to the treated patient (for example, breathing or relaxation instructions) are familiar to physicians or nurses.

Bladder instillation, also called bladder wash or bath, may help relieve inflammation, infection or repair the bladder's protective lining. During this treatment, the bladder is filled with the antimicrobial composition described herein using a catheter. The composition is held inside the bladder for a period of time ranging from 15-20 minutes. Then the composition is urinated through the urethra or drained from the bladder through the catheter. Instillation treatments may be repeated several times over a period of two to three months. Instillation of 20 to 80 mL of the composition described herein directly into the bladder may be accomplished by an aseptic syringe and allowed to remain inside the bladder for 10 to 100 minutes. The antimicrobial composition may be expelled by spontaneous voiding. It is recommended that the treatment may be repeated every week until maximum symptomatic relief is obtained. Thereafter, time intervals between treatments may be increased appropriately.

### Example 5

### Efficacy of The Antimicrobial Composition

We have devised a dynamic in vitro model using traditional microbiological methods to assess the antimicrobial efficacy of 2 mM HOCl in 0.9% saline at pH 3.5, as compared to physiological saline in disinfecting intra-luminal and extra-luminal indwelling Foley catheter.

The effectiveness of the antimicrobial composition of 2 mM HOCl in 0.9% saline at pH 3.5 on E. coli or Pr. mirabilis biofilm covered Foley catheter have been demonstrated using the materials and methods detailed below:

### Materials:

Foley Catheter, manufactured by BARD
HOCl (2 mM) in 0.9% saline pH 3.5 (150 mM)
Escherichia coli ATCC 25922
Proteus mirabilis ATCC 29245

Neutralizer Broth: A broth containing dextrose, lecithin, sodium thiosulfate, pancreatic digest of casein, Tween® 80, yeast extract, sodium bisulfate, sodium thioglycollate, monopotassium phosphate, and bromcresol purple.

Nutrient Broth and agar

### Spectrophotometer

### Methods:

The ability of HOCl to destroy biofilm formation was evaluated as follows. First, biofilm was established on 1-cm-long pieces of catheter for 48 hours in nutrient broth in the presence of either Proteus mirabilis or Escherichia coli. Subsequently, the biofilm-bearing pieces of catheter were exposed to 2 mM HOCl in 0.9% saline at pH 3.5 over various periods of time. After the exposures the pieces of catheter were transferred into 1 mL of neutralizer broth to stop the reaction. 0.1 mL (10%) of the neutralizer broth was then plated out onto nutrient agar and the number of colonies was counted. The CFU (Colony Forming Unit) values obtained were multiplied by 10 to obtain the actual CFU/mL values per treated sample.

In order to measure the amount of live bacteria left on the pieces of catheter following treatment, the biofilm-bearing pieces of catheter were transferred into tubes containing fresh growth medium. After allowing growth in a shaker at 37° C. for 4 hours, Optical Density (OD) was read at 600 nm.

Results are shown in the tables below. Cases where data were not collected are indicated by n.d.

Escherichia coli 25922

| Duration of | HOCl; 2 mM | Saline 0.9% |
|---|---|---|
| exposure | pH 3.5 | pH 3.5 |
| Minutes | CFU/mL | CFU/mL |
| <1 | 50 | >>3000 |
| 5 | 460 | n.d. |
| 10 | 0 | n.d. |
| 20 | 0 | n.d. |
| 30 | 0 | n.d. |
| 45 | 0 | n.d. |
| 60 | 0 | n.d. |
| 120 | 0 | >>3000 |

Results: E. coli infected sample but untreated had CFU/ml = >>3000 colonies and OD₆₀₀ = 0.60

Proteus Mirabilis 29245

| Duration of | HOCl; 2 mM | Saline 0.9% |
|---|---|---|
| exposure | pH 3.5 | pH 3.5 |
| Minutes | CFU/mL | CFU/mL |
| <1 | >>3000 | >>3000 |
| 5 | 110 | n.d. |
| 10 | 40 | n.d. |
| 20 | 50 | n.d. |
| 30 | 0 | n.d. |
| 45 | 20 | n.d. |
| 60 | 0 | n.d. |
| 120 | 90 | >>3000 |

Results: Pr. mirabilis infected but untreated had CFU/ml = >>3000 colonies and OD₆₀₀ = 0.17

Under the conditions of this study, Foley catheters infected with E. coli and Pr. mirabills for 48 hours and then treated with 2 mM HOCl in 0.9% saline at pH 3.5 were shown to have minimal recoverable CFU/mL bacteria. This was also shown by very low optical density readings (average of 0.057 OD₆₀₀ units, individual data not listed) following the attempt to re-culture the bacteria from treated catheters. By contrast, the same infected catheter treated with physiological saline resulted in no suppression, but rather significant re-growth of bacteria even as long as 120 minutes of treatment (both by viable count and by optical density). Therefore, the in vitro biofilm disinfection model described here demonstrated significant antimicrobial properties for 2 mM HOCl in 0.9% saline at pH 3.5, as compared to physiological saline.

Visual examination showed build up of biofilm on the catheter surface during infection and its subsequent removal by 2 mM HOCl in 0.9% saline at pH 3.5, but not by saline.

### REFERENCES

Anwar, H., J. L. Strap, et al. (1992). “Eradication of biofilm cells of Staphylococcus aureus with tobramycin and cephalexin.” Can J Microbiol 38(7); 618-25.
Baillie, L. (1987). “Chlorhexidine resistance among bacteria isolated from urine of catheterized patients.” J Hosp Infect 10(1): 83-6.
Cho, Y. H., S. J. Lee, et al. (2001). “Prophylactic efficacy of a new gentamicin-releasing urethral catheter in short-term catheterized rabbits.” BJU Int 87(1): 104-9.
Costerton, J. W., P. S. Stewart, et al. (1999). “Bacterial biofilms: a common cause of persistent infections.” Science 284(5418): 1318-22.
Cravens, D. D. and S. Zweig (2000). “Urinary catheter management.” Am Fam Physician 61(2): 369-76.
Darouiche, R. O., J. A. Smith, Jr., et al., (1999). “Efficacy of antimicrobial-impregnated bladder catheters in reducing catheter-associated bacteriuria: a prospective, randomized, multicenter clinical trial.” Urology 54(6): 976-81.
Donlan, R. M. and J. W. Costerton (2002). “Biofilms: survival mechanisms of clinically relevant microorganisms.” Clin Microbiol Rev 15(2); 167-93.
Galloway, A. (1997). “Prevention of urinary tract infection in patients with spinal cord injury—a microbiological review.”Spinal Cord 35(4): 198-204.
Hashmi, S., E. Kelly, et al. (2003). “Urinary tract infection in surgical patients.” Am. J. Surg 186(1): 53-6.
Maki, D. G. and P. A. Tambyah (2001). “Engineering out the risk for infection with urinary catheters.” Emerg Infect Dis 7(2): 342-7.
Morris, N. S., D. J. Stickler, et al. (1999). “The development of bacterial biofilms on indwelling urethral catheters.” World J Urol 17(6): 345-50.
Muncie, H. L., Jr., J. M. Hoopes, et al. (1989). “Once-daily irrigation of long-term urethral catheters with normal saline. Lack of benefit.” Arch Intern Med 149(2): 441-3,

Pearman, J. W., M. Bailey, et al. (1991). “Bladder instillations of trisdine compared with catheter introducer for reduction of bacteriuria during intermittent catheterisation of patients with acute spinal cord trauma.” Br J Urol 67(5): 483-90.
Saint, S. and C. E. Chenoweth (2003). “Biofilms and catheter-associated urinary tract infections.” Infect Dis Clin North Am 17(2): 411-32.
Stickler, D., R. Young, et al. (2003). “Why are Foley catheters so vulnerable to encrustation and blockage by crystalline bacterial biofilm?” Urol Res 31(5): 306-11
Stickler, D. J., G. L. Jones, et al. (2003). “Control of encrustation and blockage of Foley catheters.” Lancet 361(9367): 1435-7.
Tenke, P., C. R. Riedl, et al. (2004). “Bacterial biofilm formation on urologic devices and heparin coating as preventive strategy.” Int J Antimicrob Agents 23 Suppl 1; S67-74.
Trautner, B. W. and R. O. Darouiche (2004). “Catheter-associated infections: pathogenesis affects prevention.” Arch Intern Med 164(8): 842-50.
Trautner, B. W. and R. O. Darouiche (2004). “Role of biofilm in catheter-associated urinary tract infection.” Am J Infect Control 32(3): 177-83.
Trautner, B. W., R. A. Hull, et al. (2005). “Prevention of catheter-associated urinary tract infection.” Curr Opin Infect Dis 18(1): 37-41.

### Example 6

### Establishes an In Vitro Model for Biofilm Eradication and Prevention by HOCl

### Part A. Setup and Validation of System for Creating Biofilm In-Vitro

A test system was established which utilized size 14 Foley catheters (supplied by NovaCal), which were cut and installed into a pre-sterilized flow system (FIG. 1) using aseptic techniques. The system consists of five parallel channels, one channel per catheter. Sterile medium was supplied to the system via a flow-break, to prevent back-growth into the medium reservoir. The entire system was placed in a 37° C. incubator. After conditioning the system with artificial urine medium for 30 minutes, 2.0 ml inoculum from an overnight culture of urease positive Escherichia coli ATCC 25922 grown in artificial urine medium at 37° C. was introduced into the system via the valve closest to the flow break (bladder side of catheter). Each inoculum was tested to confirm urease production. After inoculation, the system remained under static conditions (no flow) for two hours, to allow for bacterial attachment to the catheters. Flow of artificial urine medium was then initiated and maintained at a rate of 0.75 ml/min for 3 days.

Initial experiments were conducted to evaluate consistency of biofilm formation in the model system. Viable cell counts indicated that by Day 3 biofilm was established at 10⁸ CFU/cm² throughout the length of the catheter. Day 5 and Day 7 counts remained at approximately that level. It was decided that treatment would be performed on Day 3 to prevent the possibility of biofilm detachment occurring.

Part B: Biofilm eradication by HOCl
Test articles used were:

Sterile saline

HOCl, 2 mM, pH 4, 0.9% by weight NaCl

HOCl, 20 mM, pH 4, 0.9% by weight NaCl

To demonstrate treatment efficacy: 20 ml of each treatment solution HOCl and sterile control solution were loaded into 30 ml syringes and connected to a syringe pump. Sterile sections of tubing were attached from the syringe to the valve furthest from the flow break (bag end of the catheter). This end is designated as FRONT for sampling purposes. The pump was turned on and the treatments were introduced at 2.0 ml/min for 10 minutes through the catheters. Excess medium and treatment solution was captured in a waste container. After 10 minutes, the syringe pump was turned off and the solutions were left stationary in the catheters for 30 minutes. The solutions were then withdrawn back through the catheter into the syringe, medium flow was resumed for a 30 minute rinse time and the catheters were then sampled.

For efficient sampling: Each catheter was divided into 3 segments (front, middle, end) and each segment was subsampled. One subsample was used to determine bacterial populations by plate count, another subsample was analyzed by staining with the LIVE/DEAD® Baclight™ bacterial viability kit (L7012, Molecular Probes, Oregon, USA) using confocal laser microscopy (CSLM), the third sample was imaged using scanning electron microscopy (SEM).

For viable cell counts, a 3.0 centimeter section of tubing was removed and scraped with a sterile stainless steel rod (using aseptic technique) into a tube containing 10.0 ml of sterile phosphate-buffered saline (PBS). The tubes were then sonicated for two minutes and the suspension was vortexed for one minute. The number of viable (culturable) bacteria was enumerated by serial dilution in PBS and plate counts using the spread-plate technique. Results were expressed as CFU/cm² and are calculated as follows:

$$\frac{( Mean CFU )}{( Volume Plated )} \times Dilution \times \frac{( Volume scraped into )}{( Surface Area )}$$

The surface area of the internal lumen of the catheter section scraped was determined to be 2.826 cm².

### Results and Data Interpretation:

Three treatment runs were performed on 3 catheters each. One treated with PBS or sterile 0.9% saline as control, one treated with 2 mM HOCl and one treated with 20 mM HOCl. The results are shown in Table 2. The Log (CFU/cm²) was calculated from the average of 9 treatments, consisting of 3 treatments of 3 catheter pieces for each Test Article.

TABLE 2
Biofilm eradication experiment.

| Test Article | Log (CFU/cm2) | Log Reduction |
|---|---|---|
| Saline | 9.1 | 0.0 |
| HOCl [2 mM] | 4.8 | −4.3 |
| HOCl [20 mM] | 2.5 | −6.6 |

Results indicate NovaCal's treatments appear to be effective at removing biofilm cells from contaminated catheters in a model urinary catheter system.

The Urinary Catheter Model developed at the CUE (Center for Biofilm Engineering at Montana State University http://www.erc.montana.edu) was shown to be an effective urinary catheter model test system. E. coli biofilms grew to uniform viable cell counts at approximately 10⁸ cfu/cm² in 3 days. This uniformity of biofilms grown in the five test catheters within the model allowed for the comparison of biofilms exposed to different treatment conditions in the catheters.

NovaCal's product, HOCl at 2 mM and 20 mM significantly reduced bacterial counts and the presence of biofilm (visual interpretation from images). The higher concentration of HOCl solution showed significantly more bacterial removal than the lower concentration

### Part C: Biofilm Prevention by HOCl

Test articles used were:

Sterile saline

White vinegar at 1:3 dilution with distilled water (filter sterilized).

Neomycin Prescription: 1 ml into 1000 ml of sterile saline

HOCl, 20 mM, pH 4, 0.9% by weight NaCl

For Biofilm Prevention Study following sequential steps were taken:

Day 0: The test system, as described in detail above, utilized size 14 Foley catheters, cut and installed into a pre-sterilized flow system using aseptic techniques. Sterile medium was supplied to the system via a flow-break, to prevent back-growth into the medium reservoir. The entire system was placed into a 37 AC incubator. After conditioning the system with artificial urine medium for 30 minutes, each catheter was treated with a disinfectant. 20.0 ml of each treatment solution was loaded into 30 ml syringes and connected to a syringe pump. Sterile sections of tubing were attached from the syringe to the valve furthest from the flow break (bag end of the catheter). This end is designated as FRONT for sampling purposes. The pump was turned on and the treatments were introduced at 2.0 ml/min for 10 minutes through the catheters. Excess medium and treatment solution was captured in a waste container. After 10 minutes, the syringe pump was turned off and the solutions were left stationary in the catheters for 30 minutes. The solutions were then withdrawn back through the catheter into the syringe. The catheters were then rinsed with sterile medium for 30 minutes.

On Day 0 only: An inoculum from an overnight culture of urease positive Escherichia coli ATCC 25922 grown in artificial urine medium at 37° C. was introduced into the system via the valve closest to the flow break (bladder side of catheter). Each inoculum was tested for confirmation of urease production. After inoculation, the system remained under static conditions (no flow) for two hours, to allow for bacterial attachment to the catheters. Flow of artificial urine medium was then initiated and maintained at a rate of 0.75 ml/min.

Days 1, 3 and 5: For viable cell counts, a 3.0 centimeter section of tubing was removed and scraped with a sterile stainless steel rod (using aseptic technique) into a tube containing 10.0 ml of sterile PBS. The tubes were then sonicated for two minutes and the suspension was vortexed for one minute. The number of viable (culturable) bacteria was enumerated by serial dilution in PBS and plate counts using the spread-plate technique. Results are expressed as CFU/cm² and were calculated as described in Phase One.

Days 1 and 3: After sampling, the catheters were disinfected and rinsed with sterile medium as described above.

Days 2 and 4: the catheters were disinfected and rinsed with sterile medium as described above. No samples were taken.

Day 5: Samples were taken for imaging and both ends of the catheter were sampled for viable cell count data.

TABLE 3
Biofilm prevention experiment.
Results and data interpretations

| | | | Day 5 | Day 5 |
|---|---|---|---|---|
| | | | “Front” | “End” |
| Day | Day 1 | Day 3 | (bag end) | (bladder end) |
| Saline | 7.6 | 7.3 | 7.7 | 8.0 |
| Vinegar | 5.7 | 3.4 | 4.1 | 3.9 |
| Neosporin | 5.5 | 3.2 | 3.8 | 4.5 |
| HOCl [20 mM] | 3.5 | 0.0 | 0.0 | 0.0 |

As seen in Table 3 and FIG. 3, NVC-101 appeared to inhibit biofilm formation during the 5 day duration of this experiment. NVC-101 appeared to be significantly better at inhibiting biofilm formation within the catheters compared to vinegar and Neosporin, especially by Day 5.

### Example 7

### Establishes Reduction of Bacterial Count Using HOCl in a Catheter Taken from a Patient

Test articles used were:

Sterile phosphate-buffered saline (PBS)

HOCl, 20 mM, pH 4, 0.9% by weight NaCl

Ex-Vivo Treatment of a Patient Catheter with HOCl

A Foley catheter was removed from a patient by hospital personnel and placed in a sterile bag. In the Bozeman Deaconess Hospital (BDH) lab, the outside of the catheter was wiped down with 70% ethanol. Then the catheter was aseptically cut into 3 catheter portions (bag-end, middle and patient-end). Each portion was cut into 3.0 cm long sections using a ruler and razor blades.

Three of the sections (one bag-end, one middle and one patient-end) designated as control were placed into sterile PBS. Three of the sections (one bag-end, one middle and one patient-end) were placed in 20 mM HOCl. All catheter sections were treated for 30 minutes individually in sterile glass tubes, each with sufficient solution to be immersed completely. After treatment, each 3 cm section was removed from the treatment tubes and the PBS control tubes and placed into a second glass tube containing sterile PBS for a 2 minute rinse in order to remove the treatment solution. The section was then removed from the tube and aseptically cut into 1.0 cm and 2.0 cm pieces. The 2.0 cm piece was placed in a tube containing 10 ml of sterile PBS, vortexed, sonicated and diluted for viable plate counts. The number of viable (culturable) bacteria was enumerated by serial dilution in PBS and plate counts using the spread-plate technique. Samples were plated on blood agar plates. Results will be expressed as colony-forming units/cm², CFU/cm² (calculated as 2.0 cm length×0.25 cm (radius)×3.14 (pi)=1.57 cm²). The 1.0 cm piece was placed in 4% formaldehyde solution.

Average Log CFU/cm² on catheter section after treatment

| | patient | | Bag | average | Log |
|---|---|---|---|---|---|
| | end | middle | end | 3 pieces | reduction |
| PBS | 3.07 | 3.84 | 1.88 | 2.93 | 2.40 |
| HOCl [20 mM] | 0.50 | 0.58 | 0.50 | 0.53 | |

### Results

In average, treating catheter pieces with HOCl 20 mM resulted in a 2.4 Log Reduction in bacterial growth compared to catheter pieces treated with sterile PBS.

While the present invention is disclosed with reference to certain embodiments and examples as provided herein, these embodiments and examples are intended to be simply illustrative of the embodiments and examples, and are not intended to be limiting in scope. Accordingly, various modifications and variations will be apparent to one skilled in the art; and those modifications and variations fall within the scope of the invention and also fall within the claims below. All references, including patents, papers and texts cited in this application are incorporated by reference herein in their entirety. It is understood that any aspect or feature of the present invention whether characterized as preferred or not characterized as preferred may be combined with any other aspect or feature of the invention, whether such other feature is characterized as preferred or not characterized as preferred. For example, a feature described as preferred, for example a pH range, or a specific pH for a particular composition (whether preferred or not) may be combined with another parameter (whether preferred or not), such as a specific halide salt concentration without deviating from the present invention This statement also applies to any combination of parameters, ingredients or constituents The terms “include(s)” or “comprise(s)” are used as open terms interchangeably in the text of this specification.

1. An antimicrobial treatment system comprising:
(a) a medical device, optionally including an accessory to the device, for implantation or insertion into a patient at risk of, or affected by, a microbial infection; and
(b) an aqueous antimicrobial composition comprising:
(1) a composition comprising an antimicrobially effective amount of hypochlorous acid HOCl, a source of hypochlorous acid, hypobromous acid HOBr, or a source of hypobromous acid; and
(2) at least one halide salt selected from the group consisting of sodium chloride, sodium bromide, potassium chloride, potassium bromide, magnesium chloride, magnesium bromide and mixtures thereof;
(3) the halide salt concentration ranging from at least about 4 to about 12 g/l of the aqueous composition;
(4) a pH from about 3 to about 6; and optionally
(5) a constituent member selected from the group consisting of buffering agents, calcium and magnesium chelating agents, biologically acceptable acids and/or salts thereof that are compatible with the antimicrobial treatment system, and mixtures thereof to maintain the pH at the range between about 3 and 6;
in order to prevent or treat colonization of the device by microbes, buildup of microbial biofilm on the device, or blockage of the device by the microbial biofilm.

2. The system of claim 1 wherein the antimicrobially effective amount of the hypohalous acid derived from the hypohalous acid or the hypohalous acid source is present at a concentration of about 0.1 mM to about 75 mM in the aqueous composition.

3. The system of claim 1 wherein the medical device is an invasive device selected from the group consisting of a central venous catheter, a peritoneal catheter, a hemodialysis shunt, an endotracheal tube, a surgical drain, a catheter for insertion into the bladder of a patient at risk of, or affected by, a bacterial, fungal or viral infection in or around the bladder and/or other infections in the patient's bloodstream, and optionally an accessory to the device including a port.

4. The system of claim 3 wherein the patient is at risk of, or affected by, bacteriuria or bacteremia Urinary Tract Infections (UTI), and/or Catheter-Associated Urinary Tract Infections (CAUTI).

5. The system of claim 2 wherein the hypohalous acid concentration is about 2 mM to about 20 mM in the composition.

6. The system of claim 2 wherein the hypohalous acid is hypochlorous acid.

7. The system of claim 2 wherein the halide salt concentration is about 7 to about 10 g/l.

8. The system of claim 7 wherein the halide salt concentration is about 9 g/l.

9. The system of claim 2 wherein the system further comprises a broad spectrum antibiotic agent.

10. The system of claim 3 in the form of a kit or tray adapted for antimicrobial treatment of a patient, optionally with antimicrobial treatment instructions.

11. A medical device selected from the group consisting of a central venous catheter, a peritoneal catheter, a hemodialysis shunt, an endotracheal tube, a surgical drain, a catheter for insertion into the bladder of a patient at risk of, or affected by, a bacterial, fungal or viral infection in or around the bladder and/or other infections in the patient's bloodstream and an accessory to the device optionally including a port, the device being treated with an aqueous antimicrobial composition for the treatment or prevention of general bacterial or fungal infections, bacteriuria or CAUTI or associated fungal or viral infections, the aqueous antimicrobial composition comprising:
(A) an antimicrobially effective amount of at least one hypohalous acid (HOHal, wherein Hal is chloro or bromo), or a hypohalous acid source;
(B) at least one saline component (halide salt) selected from the group consisting of sodium chloride, sodium bromide, potassium chloride, potassium bromide, magnesium chloride, magnesium bromide and mixtures thereof; the saline component (halide salt) concentration ranging from at least about 4 to about 12 g/l of the composition;
(C) wherein the pH (of the composition is about 3 to about 6; and
(D) the antimicrobially effective amount of the hypohalous acid derived from the hypohalous acid or the hypohalous acid source is about 0.1 mM to about 75 mM of the composition; and optionally
(E) a constituent member selected from the group consisting of buffering agents, calcium and magnesium chelating agents, biologically acceptable acids and salts thereof that are compatible with the antimicrobial treatment system, and mixtures thereof to maintain the pH between about 3 and 6
in order to prevent or treat colonization of the device by microbes, buildup of microbial biofilm on the device, or blockage of the device by the microbial biofilm.

12. The device of claim 11 wherein the antimicrobially effective amount of the hypohalous acid derived from the hypohalous acid or the hypohalous acid source is about 2 mM to about 20 mM.

13. The device of claim 11, wherein the halide salt concentration is about 7 to about 10 g/l.

14. The device of claim 13, wherein the halide salt concentration is about 9 g/l.

15. A medical device selected from the group consisting of a central venous catheter, a peritoneal catheter, a hemodialysis shunt, an endotracheal tube, a surgical drain, a catheter for insertion into the bladder of a patient at risk of, or affected by, a bacterial, fungal or viral infection in or around the bladder and/or other infections in the patient's bloodstream and an accessory to the device optionally including a port, the device being treated with an aqueous antimicrobial composition for the treatment or prevention of a general bacterial infection including bacteriuria or CAUTI or bacteremia or associated fungal or viral infections, the composition comprising:
(A) an antimicrobially effective amount of at least one hypohalous acid (HOHal), wherein Hal is chloro or bromo, or a hypohalous acid source;
(B) at least one saline component (halide salt) selected from the group consisting of sodium chloride, sodium bromide, potassium chloride, potassium bromide, magnesium chloride, magnesium bromide and mixtures thereof, the halide salt concentration ranging from at least about 4 to about 12 g/l of the aqueous composition;
(C) wherein the pH of the composition is about 3 to about 6; and
(D) the antimicrobially effective amount of the hypohalous acid or the hypohalous acid derived from the hypohalous acid source ranging from about 0.1 mM to about 75 mM in the aqueous composition; and
(E) a constituent member selected from the group consisting of buffering agents, calcium and magnesium chelating agents biologically acceptable acids and salts thereof that are compatible with the antimicrobial treatment system, and mixtures thereof to maintain the pH at the range between about 3 and 6;
in order to prevent or treat colonization of the device by microbes, buildup of microbial biofilm on the device, or blockage of the device by the microbial biofilm.

16. The device of claim 15 wherein the antimicrobially effective amount of the hypohalous acid or derived from the hypohalous acid source ranges from about 2 mM to about 20 mM.

17. The device of claim 15, wherein the halide salt concentration is about 7 g/l to about 10 g/l.

18. The device of claim 17 wherein the halide salt concentration is about 9 g/l.

19. A method of treating, inhibiting or preventing an antimicrobial infection in or near a medical device before or after said device has been inserted into a patient or a method of treating inhibiting or preventing bacterial, viral or fungal infection in a patient after said device has been inserted into a patient which comprises the following treatment steps in isolation or in combination:
(a) contacting the device with a composition comprising elements (A) through (D), and optionally (E), of claim 11, prior to insertion into a patient or after removal from a patient;
(b) washing, bathing or flushing the device with a composition comprising elements (A) through (D), and optionally (E), of claim 11, prior to insertion into a patient or after removal from a patient;
(c) irrigating the device with a composition comprising elements (A) through (D), and optionally (E), of claim 11, after insertion into a patient, in order to prevent or treat colonization of the device by microbes, buildup of microbial biofilm on the device, or blockage of the device by the microbial biofilm on the device; or
(d) instilling through the device a composition comprising components elements (A) through (D), and optionally (E), of claim 11 into the bladder of a patient to treat or prevent a fungal or bacterial infection of the lining of the bladder.

20. The method of claim 19, wherein the halide salt concentration ranges from about 7 g/l to about 10 g/l.

21. The method of claim 20, wherein the halide salt concentration is about 9 g/l.

22. The use of a composition comprising elements (A) through (D), and optionally, (E) of claim 11 for a treatment in accordance with claim 19.

23. A kit or tray comprising elements (A) through (D), and optionally (E), of claim 11; optionally with instructions for using the kit or tray in a treatment in accordance with claim 19.

24. An aqueous antimicrobial composition for the treatment or prevention of a microbial infection in patient, said composition comprising:
(a) an antimicrobially effective amount of a hypohalous acid (HOHal, wherein Hal is chloro or bromo) or a hypohalous acid source;
(b) at least one saline component (halide salt) selected from the group consisting of sodium chloride, sodium bromide, potassium chloride, potassium bromide, magnesium chloride, magnesium bromide and mixtures thereof; the saline component (halide salt) concentration is at least about 4 to about 12 g/l of the aqueous composition;
(c) a pH of about 3 to about 6; and
(d) a constituent member selected from the group consisting of buffering agents, calcium and magnesium chelating agents, biologically acceptable acids and/or salts thereof that are compatible with the antimicrobial composition, and mixtures thereof to maintain the pH at the range between about 3 and 6.

25. The composition of claim 24 wherein the antimicrobially effective amount of the hypohalous acid or the hypohalous acid derived from the hypohalous acid source is about 0.1 mM to about 75 mM in the aqueous composition.

26. The composition of claim 24 wherein the halide salt concentration is about 7 to about 10 g/l of the composition.

27. The composition of claim 26 wherein the halide salt concentration is about 9 g/l.
